(19) United States
(12) Patent Application Publication
(10) Pub. No.: US 2025/0249255 A1
(43) Pub. Date: Aug. 7, 2025

(54) SYSTEM AND METHOD FOR SEIZURE DETECTION AND VAGUS NERVE STIMULATION

(71) Applicant: THE ALFRED E. MANN FOUNDATION FOR SCIENTIFIC RESEARCH, VALENCIA, CA (US)

(72) Inventor: Jason GOLDBERG, Oakland, CA (US)

(21) Appl. No.: 19/043,095

(22) Filed: Jan. 31, 2025

Related U.S. Application Data
(60) Provisional application No. 63/548,739, filed on Feb. 1, 2024.

(51) Int. Cl. A61N 1/36

(52) U.S. Cl. CPC A61N 1/36139; A61N 1/36053; A61N 1/36064

(57) ABSTRACT

A system and method for seizure detection and vagus nerve stimulation. In some embodiments, a system includes a motion sensor configured to be secured to a subject, and a processing circuit. The processing circuit may be configure to determine a calculated heart rate of the subject based on a signal from the motion sensor, and to determine whether a seizure is imminent or occurring, based on the calculated heart rate and on a subject-specific classifier.

### CROSS-REFERENCE TO RELATED APPLICATION(S)

The present application claims priority to and the benefit of U.S. Provisional Application No. 63/548,739, filed Feb. 1, 2024, entitled “SYSTEMS AND METHODS FOR SEIZURE DETECTION IN CLOSED-LOOP VAGUS NERVE STIMULATION”, the entire content of which is incorporated herein by reference.

### FIELD

One or more aspects of embodiments according to the present disclosure relate to seizure detection and mitigation, and more particularly to a system and method for seizure detection and vagus nerve stimulation.

### BACKGROUND

Epileptic seizures may occur in subjects with epilepsy with little warning, and may result in undesirable sequelae.

It is with respect to this general technical environment that aspects of the present disclosure are related.

### SUMMARY

According to an embodiment of the present disclosure, there is provided a system, including: a motion sensor configured to be secured to a subject; and a processing circuit, the processing circuit being configured: to determine a calculated heart rate of the subject based on a signal from the motion sensor, and to determine whether a seizure is imminent or occurring, based on: the calculated heart rate; and a subject-specific classifier.

In some embodiments, the processing circuit is further configured: to determine that a seizure is imminent or occurring; and in response to the determining that a seizure is imminent or occurring, to apply closed loop vagus nerve stimulation.

In some embodiments, the subject-specific classifier includes a parameter calculator, the parameter calculator being configured to fit a calculated heart rate history with a parametric model, and to generate parameter values.

In some embodiments, the subject-specific classifier includes a seizure detector configured to determine whether a seizure is imminent or occurring, based on the parameter values.

In some embodiments, the seizure detector is configured to determine whether a seizure is imminent or occurring, based on whether a parameter value exceeds a threshold.

In some embodiments, the seizure detector includes a machine-learning classifier, configured to classify a set of one or more parameter values as corresponding to either (i) the absence of a seizure or (ii) a seizure being imminent or occurring.

In some embodiments, the machine-learning classifier is a classifier selected from the group consisting of adaptive boosting classifiers, artificial neural network learning algorithms, Bayesian belief networks, Bayesian classifiers, Bayesian neural networks, boosted trees, case-based reasoning classifiers, classification trees, convolutional neural networks, decisions trees, deep learning classifiers, elastic nets, fully convolutional networks, genetic algorithms, gradient boosting trees, k-nearest neighbor classifiers, least absolute shrinkage and selection operator classifiers, linear classifiers, naive Bayes classifiers, neural networks, logistic regression, random forests, ridge regression, support vector machines, and combinations thereof.

In some embodiments, the subject-specific classifier includes a machine-learning classifier configured to determine whether a seizure is imminent or occurring based on a calculated heart rate history.

In some embodiments, the motion sensor includes an accelerometer.

In some embodiments, the motion sensor includes a gyroscope.

According to an embodiment of the present disclosure, there is provided a method, including: determining a calculated heart rate of a subject, based on a signal from a motion sensor secured to the subject; and determining, by a subject-specific classifier, whether a seizure is imminent or occurring, based on the calculated heart rate.

In some embodiments, the method further includes: determining that a seizure is imminent or occurring; and in response to the determining that a seizure is imminent or occurring, applying closed loop vagus nerve stimulation.

In some embodiments, the subject-specific classifier includes a parameter calculator, the parameter calculator being configured to fit a calculated heart rate history with a parametric model, and to generate parameter values.

In some embodiments, the subject-specific classifier includes a seizure detector configured to determine whether a seizure is imminent or occurring, based on the parameter values.

In some embodiments, the seizure detector is configured to determine whether a seizure is imminent or occurring, based on whether a parameter value exceeds a threshold.

In some embodiments, the seizure detector includes a machine-learning classifier, configured to classify a set of one or more parameter values as corresponding to either (i) the absence of a seizure or (ii) a seizure being imminent or occurring.

In some embodiments, the machine-learning classifier is a classifier selected from the group consisting of adaptive boosting classifiers, artificial neural network learning algorithms, Bayesian belief networks, Bayesian classifiers, Bayesian neural networks, boosted trees, case-based reasoning classifiers, classification trees, convolutional neural networks, decisions trees, deep learning classifiers, elastic nets, fully convolutional networks, genetic algorithms, gradient boosting trees, k-nearest neighbor classifiers, least absolute shrinkage and selection operator classifiers, linear classifiers, naive Bayes classifiers, neural networks, logistic regression, random forests, ridge regression, support vector machines, and combinations thereof.

In some embodiments, the subject-specific classifier includes a machine-learning classifier configured to determine whether a seizure is imminent or occurring based on a calculated heart rate history.

In some embodiments, the method further includes training the subject-specific classifier.

In some embodiments, the training of the subject-specific classifier includes training the subject-specific classifier with labeled training data.

In some embodiments, the labeled training data includes a set of positive data records and a set of negative data records, each element of the set of positive data records being labeled as positive, and each element of the set of negative data records being labeled as negative.

In some embodiments, each element of the set of positive data records is further labeled with a time at which a seizure started.

In some embodiments, the motion sensor includes an accelerometer.

In some embodiments, the motion sensor includes a gyroscope.

According to an embodiment of the present disclosure, there is provided a system, including: a motion sensor configured to be secured to a subject; and a processing circuit, the processing circuit being configured to determine whether a seizure is imminent or occurring, based on: a signal from the motion sensor; and a subject-specific classifier, the subject-specific classifier including a machine learning model selected from the group consisting of fully convolutional networks, recurrent neural networks, and combinations thereof.

In some embodiments, the subject-specific classifier includes a long short-term memory neural network.

In some embodiments, the processing circuit is further configured: to determine that a seizure is imminent or occurring; and in response to the determining that a seizure is imminent or occurring, to apply closed loop vagus nerve stimulation.

In some embodiments, the subject-specific classifier includes a parameter calculator, the parameter calculator being configured to fit a calculated heart rate history with a parametric model, and to generate parameter values.

In some embodiments, the subject-specific classifier includes a seizure detector configured to determine whether a seizure is imminent or occurring, based on the parameter values.

In some embodiments, the seizure detector is configured to determine whether a seizure is imminent or occurring, based on whether a parameter value exceeds a threshold.

In some embodiments, the seizure detector includes a machine-learning classifier, configured to classify a set of one or more parameter values as corresponding to either (i) the absence of a seizure or (ii) a seizure being imminent or occurring.

In some embodiments, the machine-learning classifier is a classifier selected from the group consisting of adaptive boosting classifiers, artificial neural network learning algorithms, Bayesian belief networks, Bayesian classifiers, Bayesian neural networks, boosted trees, case-based reasoning classifiers, classification trees, convolutional neural networks, decisions trees, deep learning classifiers, elastic nets, fully convolutional networks, genetic algorithms, gradient boosting trees, k-nearest neighbor classifiers, least absolute shrinkage and selection operator classifiers, linear classifiers, naive Bayes classifiers, neural networks, logistic regression, random forests, ridge regression, support vector machines, and combinations thereof.

In some embodiments, the subject-specific classifier includes a machine-learning classifier configured to determine whether a seizure is imminent or occurring based on a calculated heart rate history.

In some embodiments, the motion sensor includes an accelerometer.

In some embodiments, the motion sensor includes a gyroscope.

### BRIEF DESCRIPTION OF THE DRAWINGS

These and other features and advantages of the present disclosure will be appreciated and understood with reference to the specification, claims, and appended drawings wherein:

FIG. 1A is a block diagram of an implantable device, according to an embodiment of the present disclosure;

FIG. 1B is a block diagram of motion sensors and associated circuits, according to an embodiment of the present disclosure;

FIG. 2A is a graph of an example of a parametrized model of ictal tachycardia, according to an embodiment of the present disclosure;

FIG. 2B is a graph of an example of a parametrized model of ictal apnea, according to an embodiment of the present disclosure;

FIG. 3 is a block diagram of a system or method for seizure detection and closed loop vagus nerve stimulation, according to an embodiment of the present disclosure; and

FIG. 4 is a block diagram of a system or method for seizure detection and closed loop vagus nerve stimulation, according to an embodiment of the present disclosure.

### DETAILED DESCRIPTION

The detailed description set forth below in connection with the appended drawings is intended as a description of exemplary embodiments of a system and method for seizure detection and vagus nerve stimulation provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

Epilepsy is a group of neurological disorders characterized by recurrent seizures. A patient, or “subject” with epilepsy may experience seizures at unpredictable times, either with no identifiable trigger, or, in some cases, triggered by an external stimulus. During a seizure, or when a seizure is imminent, the subject may exhibit an increase in heart rate, referred to as ictal tachycardia, or a decrease in respiration rate, which may be referred to as ictal apnea, or abrupt muscle contractions or spasms, or a combination of such symptoms.

Seizures (e.g., clonic seizures) may be mitigated using vagus nerve stimulation. Vagus nerve stimulation may involve applying an electrical stimulus to the vagus nerve; this stimulus may propagate to the brain and lessen the severity of a seizure, prevent a seizure, or stop a seizure that is in progress. For example, in an intervention referred to as open loop vagus nerve stimulation, the vagus nerve is periodically stimulated (e.g., for 5 seconds every 30 seconds) in an ongoing manner. In an intervention referred to as on-demand vagus nerve stimulation, the vagus nerve is stimulated in response to user input, e.g., a button press or a screen click performed by the subject or by an assistant or a clinician. In an intervention referred to as closed-loop vagus nerve stimulation, the vagus nerve is stimulated automatically (e.g., without user intervention) in response to the detection, by a control system, that a seizure has started or is imminent.

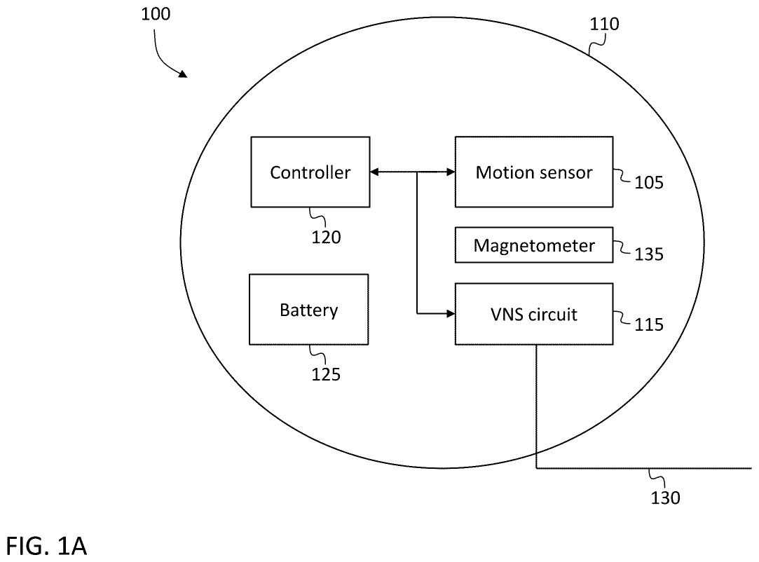

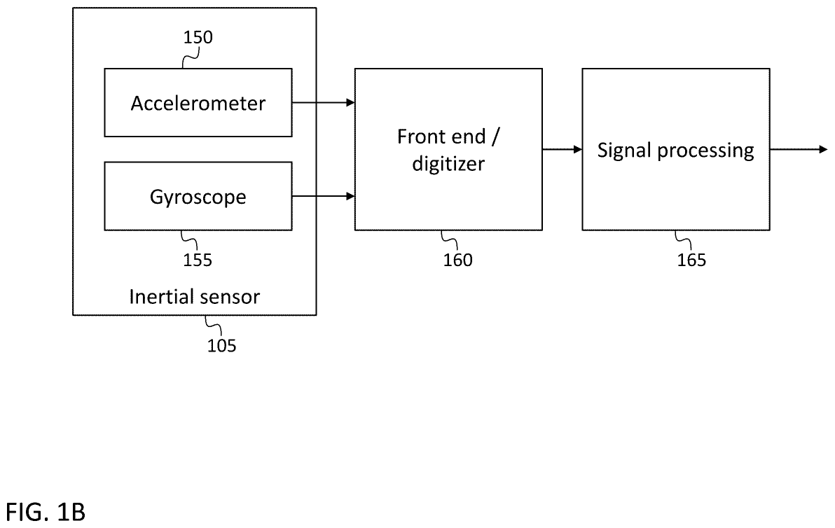

Closed-loop vagus nerve stimulation may be performed by an implantable device, such as the implantable device 100 illustrated in FIG. 1A, which may be implanted in the chest or neck of the subject. In some embodiments, a motion sensor 105 in the implantable device 100 may be employed for the detection of seizures. The motion sensor 105 may include, for example, one or more accelerometers (e.g., a three-axis accelerometer, including three accelerometers, measuring acceleration along three different axes (e.g., along three orthogonal axes)), or one or more gyroscopes (e.g., a three-axis gyroscope, including three gyroscopes, measuring rotation rate along three different axes (e.g., along three orthogonal axes)). As used herein, a “motion sensor” is a device that senses motion. As such, a single accelerometer is a motion sensor, and a set of three orthogonal accelerometers is also a motion sensor (and, as illustrated by this example, a motion sensor may include one or more motion sensors). The motion sensor 105 may be packaged in a housing 110 (discussed in further detail below) along with other components of the implantable device 100.

The motion sensor may produce signals from which various biomarkers may be calculated. For example, the motion sensor may be used to generate so-called seismocardiograms or gyrocardiograms from which instantaneous heart rate (HR) may be calculated. The same sensor signals may be used to calculate the instantaneous respiration rate (RR) as well. One exemplary method for extracting the respiration rate is to low-pass filter the inertial sensor signals, since motion due to breathing happens on a longer time scale than mechanical vibrations due to the heart. A neck mounted or implanted motion sensor may be used to measure pulsing of blood through the carotid artery. As used herein, a “biomarker” is an indicator of one or more aspects of the biological state or condition of the subject. As such, a biomarker may be the subject's heart rate, or a biomarker may be the subject's heart rate and respiration rate.

Each of the accelerometers and gyroscopes may be a microelectromechanical systems (MEMS) sensor, e.g., fabricated on a semiconductor chip using photolithographic or analogous methods, and including, e.g., a cantilevered beam that may deflect when the motion sensor 105 is subject to acceleration, or a resonant structure within which energy may couple between different resonant modes when the motion sensor 105 is rotated.

In some embodiments, the motion sensor 105 is packaged with (e.g., in the same housing 110 as) a circuit for performing vagus nerve stimulation (or VNS circuit) 115. The housing 110 may be implantable (e.g., it may be hermetically sealed and its outer surface may be composed of a biocompatible material). The implantable device 100 may include (i) the motion sensor 105 (ii) the vagus nerve stimulation circuit 115, (iii) a controller 120, which may be or include a processing circuit (discussed in further detail below) for receiving signals from or controlling the other circuits of the implantable device 100, (iv) a battery 125 for powering the circuits of the implantable device 100 and (v) the housing 110. One or more electrode wires 130 may extend from the implantable device 100 and near (e.g., the electrode wires 130 may have an end wrapped around) the vagus nerve of the subject, for providing vagus nerve stimulation. The battery 125 may be rechargeable (e.g., using power inductively coupled to the implantable device 100 through the skin of the user) or it may be a single-use battery that is replaced as needed (e.g., when the entire implantable device 100 is replaced). In some embodiments, a portion of the system may be external to the subject, e.g., secured to the chest of the subject with a strap or adhesive patch. The external portion of the system may include some or all of the elements of FIGS. 1A and 1B except for (i) the electrode wires 130, or except for (ii) the electrode wires 130 and an electrode drive circuit (which may be implanted and which may communicate with other circuitry external to the subject through an inductively coupled (signal or power) connection). The implantable device 100 may include a magnetometer 135, which may be used, for example, to detect strong magnetic fields the presence of which may increase the likelihood that a seizure will occur.

The blocks illustrated in FIG. 1A may be implemented in hardware or in some combination of hardware, software, and firmware. For example, each of the motion sensor 105 and the magnetometer 135 may include analog sensors and analog-to-digital converters, and the vagus nerve stimulation circuit 115 may include a digital-to-analog converter for converting a digital signal (received by, or generated by, the vagus nerve stimulation circuit 115) to a suitable analog stimulation signal for the vagus nerve.

FIG. 1B shows an inertial sensor 105 including an accelerometer 150 (e.g., a three-axis accelerometer) and a gyroscope 155 (e.g., a three-axis gyroscope), connected to a front end and digitizer circuit 160 (which may include one or more analog preamplifiers and one or more analog to digital converters) and a signal processing circuit 165 (which may be a processing circuit (discussed in further detail below)). A system like that shown in FIG. 1B, or a portion of such a system (e.g., the inertial sensor 105) may be secured to the chest of a subject, or implanted in the subject (or a portion of it may be implanted in the subject), and used to obtain seismocardiograms or gyrocardiograms.

Automatic detection of seizures based on instantaneous heart rate calculations may be used to initiate stimulation in epileptic patients with implanted closed-loop, vagus nerve stimulation devices. In particular, hypothesis testing may be applied to sequential heart rate calculations to detect the presence of ictal tachycardia. Such detection strategies may involve computation of a test statistic and the comparison of the test statistic to some threshold.

Changes in instantaneous heart rate due to ictal tachycardia may be subject-specific, e.g., may vary in form from subject to subject. For example, the total increase in heart rate or the instantaneous rate of increase may be subject-specific. As such, some embodiments employ personalized, per-patient seizure detection, which may test for particular forms of heart increase characteristically associated with an individual patient. Testing of this type may lead to improved receiver operating characteristic (ROC) or lower latency. For example, the characteristics of multiple ictal-tachycardia-related instantaneous heart rate increases may be observed for each subject, and the observations may be used to create a subject-specific system or method for detecting that a seizure is imminent or occurring.

In some embodiments, the average heart rate history during ictal tachycardia is calculated, e.g., by averaging together multiple heart rate histories (e.g., calculated heart rate histories) corresponding to multiple respective occurrences of ictal tachycardia. The current heart rate history (e.g., calculated heart rate history) of the subject may be compared, continuously or periodically, (i) to this average heart rate history during ictal tachycardia, and (ii) to a set of heart rate histories when ictal tachycardia is not occurring (or a statistical characterization of such a set) to determine whether a seizure is imminent or occurring. Such a comparison may include calculating a measure of similarity between the current heart rate history and the average heart rate history during ictal tachycardia. The calculation may be performed for example, by fitting the average heart rate history during ictal tachycardia with a parametric model to obtain a set of reference parameter values, fitting the current heart rate history of the subject with the parametric model to obtain a set of current parameter values, and comparing the current parameter values to the reference parameter values. As used herein, a heart rate history (or calculated heart rate history) is a sequence of calculations of the heart rate, extending over some interval of time (e.g., backward, from the present, to a point in the past). The interval may have a length of between 5 seconds and 6000 seconds.

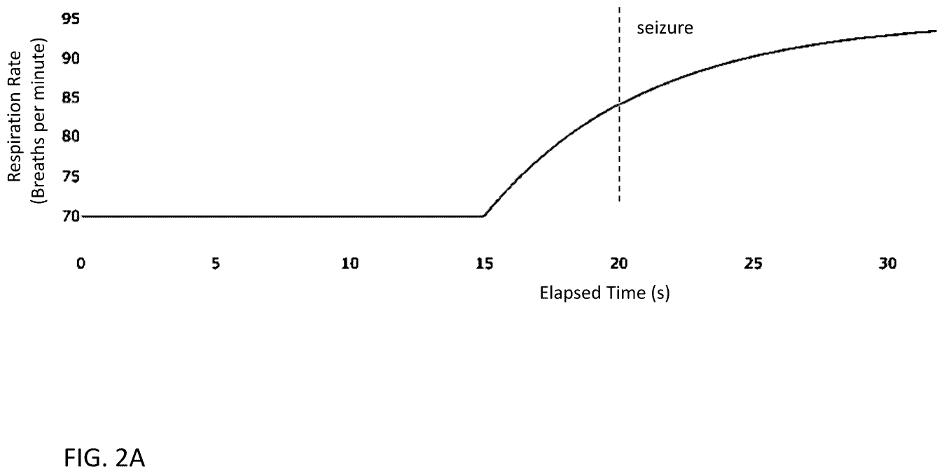

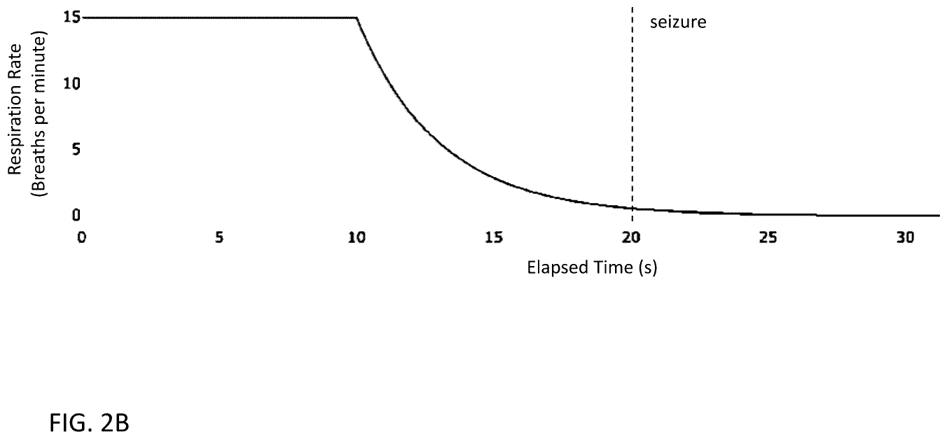

FIG. 2A shows a graph of one example of a parametrized model of ictal tachycardia; in this model the heart rate is modeled as following an exponential function, which is parametrized by a start time, an amplitude (of 25 beats per minute, in the example of FIG. 2A), and a time constant (of 6 seconds, in the example of FIG. 2A). For such a model, a modest increase in calculated heart rate (e.g., due to a slight increase in exertion in the subject, or due to noise in the motion sensor 105) may result in the current heart rate history being best fit by a set of parameters in which the amplitude is small, or the time constant is large. As such, the system may continuously or periodically compare the amplitude and time constant corresponding to the current heart rate history to respective thresholds, and determine that a seizure is imminent or occurring if both (i) the amplitude exceeds the threshold amplitude and (ii) the time constant is less than the threshold time constant. In some embodiments, the ratio of the amplitude to the time constant (a ratio that may be proportional to the maximum rate of change, in the model) is compared to a threshold and the system determines that a seizure is imminent or occurring if the ratio exceeds a threshold.

FIG. 2B shows a graph of an exponential model of respiration rate that may be employed in an analogous manner to determine whether a measured change in the respiration rate is ictal apnea, e.g., whether a seizure is imminent or occurring. In the model shown in FIG. 2B, the respiration rate is modeled as following an exponential function, which is parametrized by a start time, an amplitude (of 15 breaths per minute, in the example of FIG. 2B), and a time constant (of 3 seconds, in the example of FIG. 2B). As in the example of FIG. 2A, the system may determine that a seizure is imminent or occurring if both (i) the amplitude, in the model, exceeds a threshold amplitude and (ii) the time constant, in the model, is less than the threshold time constant, or if the ratio of the amplitude to the time constant exceeds a threshold. In some embodiments, changes in heart rate and changes in respiration rate are jointly taken into account when a determination is made as to whether a seizure is imminent or occurring. In each of FIG. 2A and FIG. 2B, a dashed line shows a point in time at which a seizure may have begun.

In some embodiments, a different parametrized model of the heart rate is used. Such a model may be any function or representation that, when fit to a heart rate history, results in parameter values that may be used to determine whether a seizure is imminent or occurring (e.g., parameter values that depend on whether a seizure is imminent or occurring). For example, a parametrized model may be a polynomial model, a frequency-domain model, a wavelet domain model, or a matrix-based model such as empirical mode decomposition. In some embodiments, the system uses a model based on an average waveform that is an average of heart rate histories previously observed for the subject during a seizure. In such an embodiment, the parameters may be (or include) an amplitude (an overall scale factor by which the average waveform is multiplied) and a time offset.

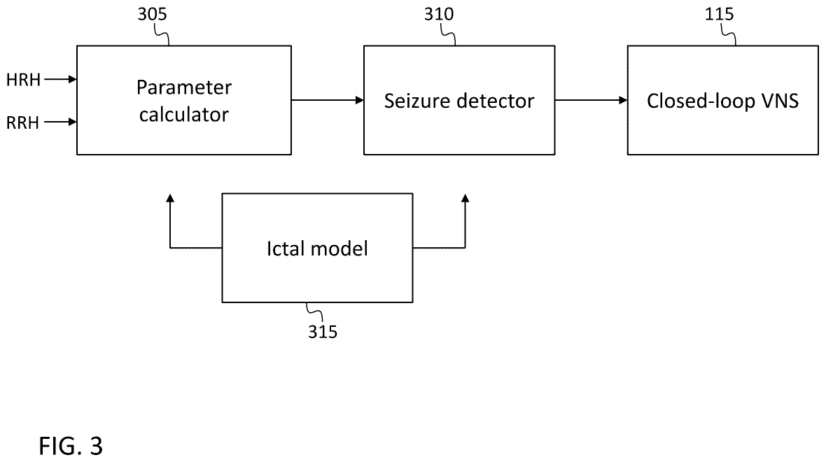

FIG. 3 is a block diagram of a system or method for determining whether a seizure is imminent or occurring and for performing closed-loop vagus nerve stimulation. A parameter calculator 305 (which may also be referred to as a feature generator) receives a calculated heart rate history (HRH) or a calculated respiration rate history (RRH) and fits a parametrized model to at least one of the one or more histories it receives, to generate a set of one or more parameter values. This may be repeated periodically, e.g., once per second. The parameter values are fed to a seizure detector 310, which makes a determination, e.g., for each set of parameter values, of whether a seizure is imminent or occurring. Each of the parameter calculator 305 and the seizure detector 310 may be implemented in software (e.g., firmware running on the controller 120), or in hardware (e.g., in a dedicated circuit, or using a general-purpose processing circuit separate from the controller 120), or in a combination of software (e.g., firmware) and hardware.

The seizure detector 310 may make this determination based on any combination of the parameter values it receives. For example, the seizure detector 310 may make the determination based on parameter values of the heart rate model, or based on parameter values of the respiration rate model (or on both). In an embodiment in which each of the heart rate model and the respiration rate model is an exponential function (as discussed in the context of FIG. 2A and FIG. 2B), the seizure detector 310 may for example, determine that a seizure is imminent or occurring (i) if the amplitude of the heart rate model exceeds a first threshold and the time constant of the heart rate model is less than a second threshold or (ii) if the amplitude of the respiration rate model exceeds a third threshold and the time constant of the respiration rate model is less than a fourth threshold or (iii) if both (i) and (ii) are met, e.g., if each amplitude exceeds a respective threshold and each time constant is less than a respective threshold. The thresholds may be determined based on (i) training (as discussed in further detail below), and otherwise fixed, or on (ii) other parameter values. For example the third and fourth thresholds may be determined based on the parameter values of the heart rate model (with, e.g., the third and fourth thresholds being lower and higher, respectively, if the ratio of the heart rate model amplitude to the heart rate model time constant is greater). When the seizure detector 310 determines that a seizure is imminent or occurring, it may send a signal (e.g., a command) to the vagus nerve stimulation circuit 115 to cause the vagus nerve stimulation circuit 115 to perform vagus nerve stimulation.

The multiple sensors of the implantable device 100 of FIG. 1A may be used to identify scenarios associated with higher risk of seizure. For example, if it is found, for a particular subject, that seizures are more likely at a certain time of day (e.g., in the morning) or in the presence of a strong magnetic field, such information may be reported to the subject. Moreover, when such conditions are encountered, an elevated risk warning may be communicated to the subject, or a detection threshold may be modified

In some embodiments, the seizure detector 310 is or includes a machine-learning model, e.g., a machine-learning classifier, such as an adaptive boosting classifier (AdaBoost), an artificial neural network (ANN) learning algorithm, a Bayesian belief network, a Bayesian classifier, a Bayesian neural network, a boosted tree, a case-based reasoning classifier, a classification tree, a convolutional neural network, a decisions tree, a deep learning classifier, an elastic net, a fully convolutional network (FCN), a genetic algorithm, a gradient boosting tree, a k-nearest neighbor classifier, a least absolute shrinkage and selection operator (LASSO) classifier, a linear classifier, a naive Bayes classifier, a neural network, logistic regression (e.g., penalized logistic regression), a random forest, ridge regression, a support vector machine, or a combination thereof. Such a machine-learning classifier may be trained (e.g., using supervised training) as discussed in further detail below.

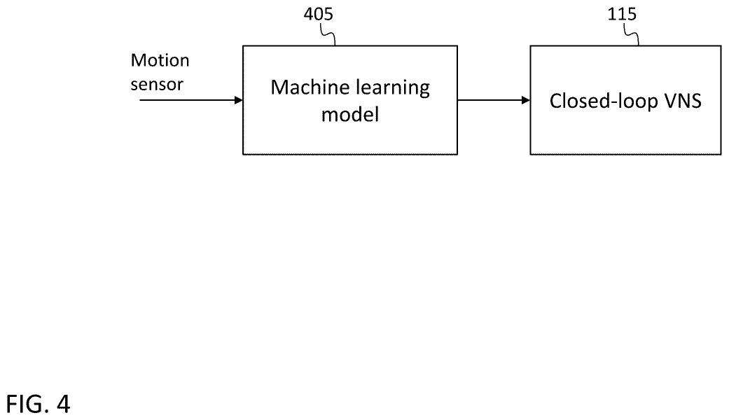

In some embodiments, the parameter calculator 305 and the seizure detector 310 are both machine-learning models. In such an embodiment the combination of the parameter calculator 305 and the seizure detector 310 may be considered to be a single (composite) machine-learning model, with an internal feature map that includes (e.g., consists of) the calculated heart rate of the subject. In some embodiments, a single machine-learning model performs the functions of the combination of the parameter calculator 305 and the seizure detector 310. Such a machine-learning model need not have an internal boundary between a first portion that performs parameter calculation and a second portion that performs seizure detection, at which parameter values of a parametrized model for the calculated heart rate is transmitted from the first portion to the second portion; instead (i) latent variables that form the output of the first portion may be, for example, linear combinations of (or nonlinear functions of) the calculated heart rate and the calculated respiration rate, or (ii) (as illustrated in FIG. 4) the machine-learning model may map detected motion (e.g., the raw signals from motion sensors) directly to a determination of whether a seizure is imminent or occurring without using any internal (latent) variables that correspond directly to heart rate or respiration rate. In the latter embodiment (in which the machine-learning model may map detected motion directly to a determination of whether a seizure is imminent or occurring), or in other embodiments disclosed herein, machine learning algorithms that may offer attractive performance when trained on imbalanced data sets (e.g., data sets including far more data without seizure than with) and accept time series data (e.g., raw multi-dimensional time series data) include convolutional neural networks (e.g., fully convolutional networks) and recurrent neural networks (e.g., long short-term memory (LSTM) approaches). In any such embodiment the determination of whether a seizure is imminent or occurring may be used to perform closed-loop vagus nerve stimulation (as illustrated, for example, in FIGS. 3 and 4), with, for example, closed-loop vagus nerve stimulation being performed in response to a determination that a seizure is imminent or occurring.

Some methods described herein may be performed by the implantable device 100 (e.g., by the controller 120 of the implantable device 100). In some embodiments, some methods (e.g., the training of one or more machine-learning models) may be performed by other computing systems (e.g., to reduce the computational burden on the controller 120). Such other computing systems may include a computing system connected directly to the implantable device 100 (e.g., through a wireless connection such as Bluetooth™) or computing systems (e.g., a server connected to the internet) connected indirectly to the implantable device 100. For example, the implantable device 100 may be connected by a wireless connection to a mobile device (e.g., a mobile telephone, a laptop computer, or a tablet computer), which may perform some of the methods (e.g., the training of a machine-learning model) described herein, or which may relay data (e.g., training data and machine-learning model parameters (e.g., weights of a neural network)) between the implantable device 100 and another computing system (e.g., a server connected to the internet) which may perform some of the methods (e.g., the training of a machine-learning model) described herein.

Training of the machine-learning models described herein may include supervised training, and may result in a subject-specific machine-learning model (e.g., a machine-learning subject-specific classifier). For example, during a training interval (which may an interval with a length of between 5 days and 10 years (e.g., between 3 weeks and 50 weeks)) the subject may report occurrences of seizures (e.g., when the subject senses that a seizure is imminent, or when the subject becomes aware that a seizure has occurred), or the system may detect seizures by the occurrence (as measured by the motion sensor 105) of motions (e.g., shaking of the subject's torso in a way consistent with clonic seizure convulsions), that would result from muscle movements characteristic of a seizure. When a seizure is reported or detected, a record of motion sensor data (or a record of calculated heart rates or a record of calculated respiration rates) may be saved, and labeled as positive, e.g., as data corresponding to the occurrence of a seizure.

In some embodiments, such data records, each of which may be referred to as a “positive data record”, may be shifted in time (e.g., based on the time at which the seizure was reported and based on whether a recent seizure or an imminent seizure was reported) such that the start times of the seizures are approximately aligned in all of the positive data records. The positive data records may then be truncated at the beginning or at the end, such that all of the positive data records have approximately the same length and all of the positive data records include data corresponding to a time interval during which a seizure began at approximately the same time within each interval. In other embodiments, each positive data record corresponds to a time interval during which a seizure occurred, and each positive data record may be labeled with the calculated time at which the seizure occurred in addition to being labeled as positive. In such an embodiment, the classifier may be trained to produce both (i) a determination of whether a seizure is imminent or occurring and (ii) a calculation of when the seizure began or will begin.

Training data corresponding to time intervals during which no seizure occurred, or “negative data records”, may be obtained by saving data for time intervals during which no seizures were reported by the subject, or during which motions that would result from muscle movements characteristic of a seizure are absent from the motion sensor data. Supervised training may be performed with the labeled training data (e.g., with the positive data records and with the negative data records), e.g., using back propagation, to produce learned parameters, or “weights” that may be stored for use by the machine-learning model. The training may be performed by the controller 120, or a more capable computing system (e.g., a computing system connected directly to the implantable device 100, or a server connected to the internet, or a plurality of servers connected to the internet) may be used to perform the training, e.g., to reduce the time required to complete the training. In some embodiments, the training is performed after a set of training data has been collected. In some embodiments, training is instead or also performed during operation. For example, the system may begin operation with an initial set of machine-learning model weights; this initial set of weights may be ones obtained by training the machine-learning model with positive data records and negative data records obtained from a plurality of other subjects. Subject-specific training data may then be collected from the subject, while the system is operating (e.g., performing seizure detection and closed-loop vagus nerve stimulation), and the weights may be modified by, or replaced with new weights obtained from, further training using the subject-specific training data.

Each of the elements or methods for performing a data processing function (e.g., for performing heart rate calculation, for performing respiration rate calculation, for performing parameter calculation, or for performing seizure detection) described herein (e.g., shown in FIG. 3) may be implemented in hardware (e.g., as one or more circuits, e.g., processing circuits) or in software, or in a combination of hardware and software.

As used herein, a “subject-specific” system or method is a system or method the behavior of which is tailored to a subject, e.g., a system or method the behavior of which is based on one or more characteristics of the subject, the characteristics being ones that are different in another subject. As such, a subject-specific classifier may be a classifier that uses parameters (e.g., thresholds) based on characteristics that vary from subject to subject (e.g., a machine-learning model trained with subject-specific training data). For example, a subject-specific classifier may be trained with data obtained during seizures experienced by the subject. To the extent that such data includes unique characteristics not found in data from any other subject, the subject-specific classifier may be unique to a particular subject. In other embodiments, a subject-specific classifier may instead be tailored to a group of subjects, e.g., a group based on the age, ethnicity, height or weight of the subject. For example, if the heart rate or respiration rate profiles, during seizures, are typically different for subjects in different age ranges, then a subject-specific classifier may be one that is trained or otherwise configured to detect a heart rate or respiration rate profile expected during seizure for the age range within which the subject is found.

Although some examples discussed herein discuss the use of an accelerometer to generate a seismocardiogram, from which a heart rate may be calculated and used for seizure detection, the present disclosure is not limited to such embodiments. For example, a gyroscope may be used in an analogous manner to generate a gyrocardiogram from which a heart rate may be calculated, or a combination of a gyroscope and an accelerometer may be used to generate a signal that responds to cardiac motion, and that may be used, for example, to calculate the heart rate. Similarly a respiration rate may be calculated from motion sensor signals (e.g., signals from one or more accelerometers or one or more gyroscopes) and used (by itself, or with the heart rate, for seizure detection.

The embodiments described herein may result in improved performance (e.g., improved receiver operating characteristic (ROC)) in detecting seizures and therefore improved performance in mitigating seizures using closed-loop vagus nerve stimulation. As such, these embodiments improve the technology of seizure detection and the technology of closed-loop vagus nerve stimulation.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B. As used herein, “approximately” includes “exactly”, and, as such, “exactly” is a special case of “approximately”.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1− 35/100) times 10) and the recited maximum value of 13.5 (i.e., (1+ 35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “directly connected” or “directly coupled” to another element, there are no intervening elements present. As used herein, “generally connected” means connected by an electrical path that may contain arbitrary intervening elements, including intervening elements the presence of which qualitatively changes the behavior of the circuit. As used herein, “connected” means (i) “directly connected” or (ii) connected with intervening elements, the intervening elements being ones (e.g., low-value resistors or inductors, or short sections of transmission line) that do not qualitatively affect the behavior of the circuit.

Although some examples discussed herein discuss the use of an accelerometer to generate a seismocardiogram, the present disclosure is not limited to such embodiments. For example, a gyroscope may be used in an analogous manner (e.g., with analogous systems and methods for reducing the effects of motion artifacts) to generate a gyrocardiogram, or a combination of a gyroscope and an accelerometer may be used to generate a signal that responds to cardiac motion, and that may be used, for example, to calculate the heart rate.

Although some examples discussed herein discuss the use of a calculated heart rate to determine whether a seizure is imminent or occurring, the present disclosure is not limited to such embodiments. For example, a calculated respiration rate may be used in an analogous manner (e.g., with analogous systems and methods for reducing the effects of motion artifacts), instead of, or in addition to, the heart rate, to detect when a seizure is imminent or occurring.

Although exemplary embodiments of a system and method for seizure detection and vagus nerve stimulation have been specifically described and illustrated herein, many modifications and variations will be apparent to those skilled in the art. Accordingly, it is to be understood that a system and method for seizure detection and vagus nerve stimulation constructed according to principles of this disclosure may be embodied other than as specifically described herein. The invention is also defined in the following claims, and equivalents thereof.

1. A system, comprising:
a motion sensor configured to be secured to a subject; and
a processing circuit,
the processing circuit being configured:
to determine a calculated heart rate of the subject based on a signal from the motion sensor, and
to determine whether a seizure is imminent or occurring, based on:
the calculated heart rate; and
a subject-specific classifier.

2. The system of claim 1, wherein the processing circuit is further configured:
to determine that a seizure is imminent or occurring; and
in response to the determining that a seizure is imminent or occurring, to apply closed loop vagus nerve stimulation.

3. The system of claim 1, wherein the subject-specific classifier comprises a parameter calculator, the parameter calculator being configured to fit a calculated heart rate history with a parametric model, and to generate parameter values.

4. The system of claim 3, wherein the subject-specific classifier comprises a seizure detector configured to determine whether a seizure is imminent or occurring, based on the parameter values.

5. The system of claim 4, wherein the seizure detector is configured to determine whether a seizure is imminent or occurring, based on whether a parameter value exceeds a threshold.

6. The system of claim 4, wherein the seizure detector comprises a machine-learning classifier, configured to classify a set of one or more parameter values as corresponding to either (i) the absence of a seizure or (ii) a seizure being imminent or occurring.

7. The system of claim 6, wherein the machine-learning classifier is a classifier selected from the group consisting of adaptive boosting classifiers, artificial neural network learning algorithms, Bayesian belief networks, Bayesian classifiers, Bayesian neural networks, boosted trees, case-based reasoning classifiers, classification trees, convolutional neural networks, decisions trees, deep learning classifiers, elastic nets, fully convolutional networks, genetic algorithms, gradient boosting trees, k-nearest neighbor classifiers, least absolute shrinkage and selection operator classifiers, linear classifiers, naive Bayes classifiers, neural networks, logistic regression, random forests, ridge regression, support vector machines, and combinations thereof.

8. The system of claim 1, wherein the subject-specific classifier comprises a machine-learning classifier configured to determine whether a seizure is imminent or occurring based on a calculated heart rate history.

9. The system of claim 1, wherein the motion sensor comprises an accelerometer.

10. The system of claim 1, wherein the motion sensor comprises a gyroscope.

11. A method, comprising:
determining a calculated heart rate of a subject, based on a signal from a motion sensor secured to the subject; and
determining, by a subject-specific classifier, whether a seizure is imminent or occurring, based on the calculated heart rate.

12. The method of claim 11, further comprising:
determining that a seizure is imminent or occurring; and
in response to the determining that a seizure is imminent or occurring, applying closed loop vagus nerve stimulation.

13. The method of claim 11, wherein the subject-specific classifier comprises a parameter calculator, the parameter calculator being configured to fit a calculated heart rate history with a parametric model, and to generate parameter values.

14. The method of claim 13, wherein the subject-specific classifier comprises a seizure detector configured to determine whether a seizure is imminent or occurring, based on the parameter values.

15. The method of claim 14, wherein the seizure detector is configured to determine whether a seizure is imminent or occurring, based on whether a parameter value exceeds a threshold.

16.-24. (canceled)

25. A system, comprising:
a motion sensor configured to be secured to a subject; and
a processing circuit,
the processing circuit being configured to determine whether a seizure is imminent or occurring, based on:
a signal from the motion sensor; and
a subject-specific classifier,
the subject-specific classifier comprising a machine learning model selected from the group consisting of fully convolutional networks, recurrent neural networks, and combinations thereof.

26. The system of claim 25, wherein the subject-specific classifier comprises a long short-term memory neural network.

27. The system of claim 25, wherein the processing circuit is further configured:
to determine that a seizure is imminent or occurring; and
in response to the determining that a seizure is imminent or occurring, to apply closed loop vagus nerve stimulation.

28. The system of claim 25, wherein the subject-specific classifier comprises a parameter calculator, the parameter calculator being configured to fit a calculated heart rate history with a parametric model, and to generate parameter values.

29. The system of claim 28, wherein the subject-specific classifier comprises a seizure detector configured to determine whether a seizure is imminent or occurring, based on the parameter values.

30.-35. (canceled)
